(19) United States
(12) Patent Application Publication
(10) Pub. No.: US 2025/0375151 A1
(43) Pub. Date: Dec. 11, 2025

(54) SURGICAL NAVIGATION SYSTEM AND METHOD OF USING SAME

(71) Applicant: CTL Amedica Corporation, Carrollton, TX (US)

(72) Inventors: Sean Suh, Paramus, NJ (US); Jon Suh, Ambler, PA (US)

(73) Assignee: CTL Amedica Corporation, Carrollton, TX (US)

(21) Appl. No.: 19/232,226

(22) Filed: Jun. 9, 2025

Related U.S. Application Data
(60) Provisional application No. 63/657,433, filed on Jun. 7, 2024.

(51) Int. Cl. A61B 5/00; A61B 5/0515

(52) U.S. Cl. CPC A61B 5/4528; A61B 5/0515

(57) ABSTRACT

A surgical navigation system including at least one tracking system having a magnetic field generator for generating a magnetic field; a joint sensor detectable within the magnetic field; an instrument motion sensor detectable within the magnetic field; and a processor. The processor is configured to receive patient-specific image data of a joint of a patient, receive position data from the joint sensor, receive position data from the instrument motion sensor, update the patient-specific image data based on one of the received position data from the joint sensor and the instrument motion sensor, and output the updated patient-specific image data. The joint sensor can include a marker including at least one of a pin and a fiber member.

### CROSS REFERENCE TO RELATED APPLICATIONS

The present application claims the benefit of U.S. Provisional Patent Application No. 63/657,433 filed Jun. 7, 2024 entitled “SPINE SURGERY NAVIGATION SYSTEM AND METHOD UTILIZING MOTION SENSORS,” the entire disclosure of which is hereby incorporated herein by reference.

### BACKGROUND OF THE INVENTION

Joint surgeries often involve operating on complex areas where nerves and blood vessels are densely concentrated. Serious complications, such as nerve damage or bleeding, can occur during such surgeries if an insertion position of a surgical instrument is not accurate. A surgical navigation system is a medical system that helps surgeons accurately identify positions of the patient's body along with positions of surgical instruments used by a surgeon by utilizing imaging technologies. Accordingly, surgical navigation systems help minimize the risks associated with such surgeries and can be used to increase the success rate of the surgeries.

Conventional surgical navigation systems include an optical imaging device, a tracking system, and surgical instruments. Such conventional surgical navigation systems can include inaccuracies from a mismatch between an image of a patient and the actual anatomy of the patient. Furthermore, due to the size of conventional optical sensors, a significant amount of space near the surgical site is occupied which can lead to further issues with accuracy of the system and user visibility. This space constraint limits the placement of multiple optical sensors on the anatomy, as it is essential to preserve visual access space.

### BRIEF SUMMARY OF THE INVENTION

A surgical navigation system is described herein. The surgical navigation system includes a tracking system having a magnetic field generator for generating a magnetic field; a joint sensor detectable within the magnetic field; an instrument motion sensor detectable within the magnetic field; and a processor. The processor is configured to receive patient-specific image data of a joint of a patient, receive position data from the joint sensor, receive position data from the instrument motion sensor, update the patient-specific image data based on one of the received position data from the joint sensor and the instrument motion sensor, and output the updated patient-specific image data.

In an aspect, each of the joint sensor and the instrument motion sensor are configured to emit a signal. In such an aspect, the processor is further configured to receive the signal emitted from at least one of the joint sensor and the instrument motion sensor, and convert the received signal emitted from the at least one of the joint sensor and the instrument motion sensor to position data.

In an aspect, the joint sensor includes a marker. In such an aspect, the marker includes at least one of a pin and a fiber member. In an aspect, the joint is a vertebrae, a knee joint, a hip joint, an elbow joint, an ankle joint, a shoulder joint, or a wrist joint. In an aspect the surgical navigation system includes a reference sensor detectable within the magnetic field for registering a frame of reference.

In an aspect, the joint sensor and the instrument motion sensor include a non-ferrous metal. In an aspect, the processor is further configured to receive image data of a surgical instrument; and update the patient-specific image data based on the received image data of the surgical instrument.

A method for performing surgical navigation as described herein includes: receiving patient-specific image data of a joint of a patient; generating a magnetic field; attaching a joint sensor detectable within the magnetic field to a bone of the joint; tracking position data of the joint sensor within the magnetic field; updating the patient-specific image data based on the tracked position data of the joint sensor; and outputting the updated patient-specific image data. In an aspect, the joint used in the method is a vertebrae.

In an aspect, the method includes attaching the joint sensor to the joint via a marker. In an aspect, the method includes attaching a plurality of joint sensors to a plurality of vertebra respectively. In an aspect, the method includes tracking position data of each of the plurality of vertebra; and updating the patient-specific image data based on the tracked position data of at least two of the plurality of vertebra. In an aspect, the method includes attaching at least three joint sensors respectively to at least three adjacent vertebra.

In an aspect, the method includes attaching a reference sensor to a predetermined vertebra of the patient, and attaching at least three joint sensors respectively to at least three vertebra adjacent the predetermined vertebra. In an aspect, the method includes receiving image data of a surgical instrument; receiving position data from an instrument motion sensor; and updating the patient-specific image data based on the received image data of the surgical instrument and the received position data from the instrument motion sensor.

### BRIEF DESCRIPTION OF THE SEVERAL VIEWS OF THE DRAWINGS

The foregoing summary, as well as the following detailed description of the exemplary embodiments of the invention, will be better understood when read in conjunction with the appended drawings. For the purpose of illustrating the invention, there are shown in the drawings exemplary embodiments. It should be understood, however, that the exemplary embodiments are not limited to the precise arrangements and instrumentalities shown.

FIG. 1A illustrates a surgical navigation system according to an exemplary embodiment of the subject disclosure;

FIG. 1B illustrates table arm sensors attached to table arms according to an exemplary embodiment of the subject disclosure;

FIG. 1C illustrates a tracking system according to an exemplary embodiment of the subject disclosure;

FIG. 2 illustrates joint sensors attached to joints of a patient according to an exemplary embodiment of the subject disclosure;

FIG. 3 illustrates joint sensors attached to joints of a patient in accordance with an alternative aspect of the subject disclosure;

FIG. 4 illustrates a digital representation of joint sensors attached to joints of patient;

FIG. 5 illustrates another digital representation of joint sensors attached to joints of a patient;

FIG. 6 illustrates a digital representation of an instrument sensor and joint sensors attached to joints of patient;

FIG. 7 illustrates yet another digital representation of joint sensors attached to joints of a patient;

FIG. 8 is a schematic diagram of a surgical navigation system in accordance with an exemplary embodiment of the subject disclosure;

FIG. 9 illustrates a surgical navigation method according to an exemplary embodiment of the subject disclosure; and

FIG. 10 illustrates a surgical navigation method according to another exemplary embodiment of the subject disclosure.

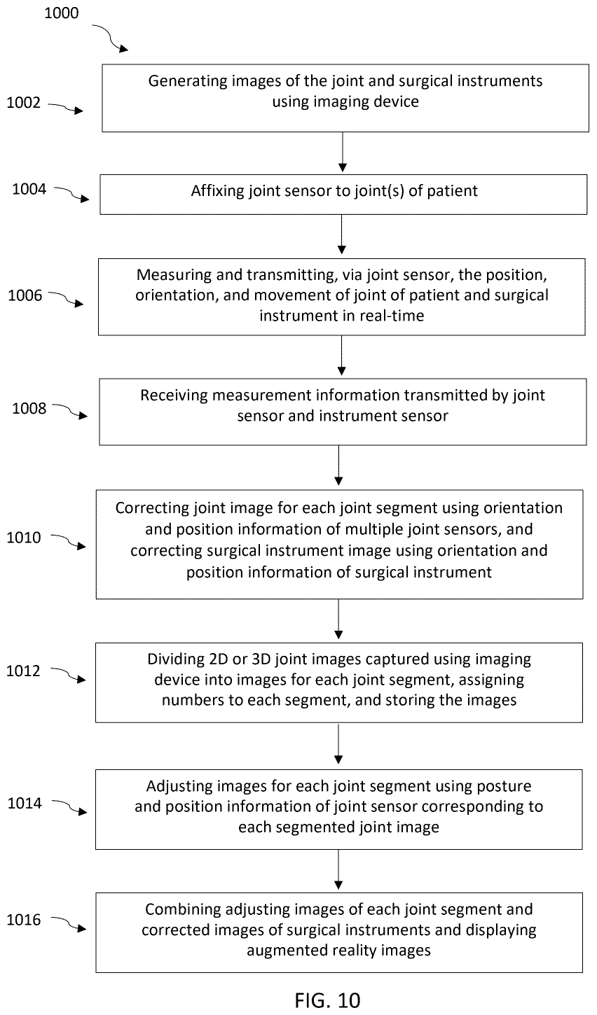

### DETAILED DESCRIPTION OF THE INVENTION

Reference will now be made in detail to the various exemplary embodiments of the subject disclosure illustrated in the accompanying drawings. Wherever possible, the same or like reference numbers will be used throughout the drawings to refer to the same or like features. It should be noted that the drawings are in simplified form and are not drawn to precise scale. Certain terminology is used in the following description for convenience only and is not limiting. Directional terms such as top, bottom, left, right, above, below and diagonal, are used with respect to the accompanying drawings. The term “distal” shall mean away from the center of a body. The term “proximal” shall mean closer towards the center of a body and/or away from the “distal” end. The words “inwardly” and “outwardly” refer to directions toward and away from, respectively, the geometric center of the identified element and designated parts thereof. Such directional terms used in conjunction with the following description of the drawings should not be construed to limit the scope of the subject disclosure in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of +20%, +10%, +5%, +1%, or +0.1% from the specified value, as such variations are appropriate.

“Substantially” as used herein shall mean considerable in extent, largely but not wholly that which is specified, or an appropriate variation therefrom as is acceptable within the field of art. “Exemplary” as used herein shall mean serving as an example.

Throughout this disclosure, various aspects of the subject disclosure can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages and characteristics of the exemplary embodiments of the subject disclosure may be combined in any suitable manner in one or more embodiments. One skilled in the relevant art will recognize, in light of the description herein, that the present disclosure can be practiced without one or more of the specific features or advantages of a particular exemplary embodiment.

In other instances, additional features and advantages may be recognized in certain embodiments that may not be present in all exemplary embodiments of the subject disclosure.

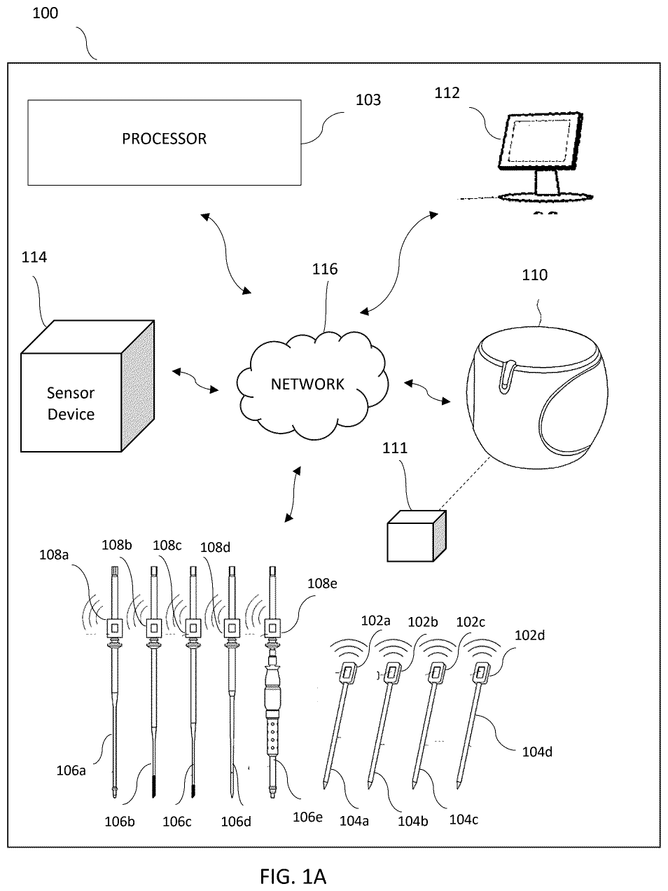

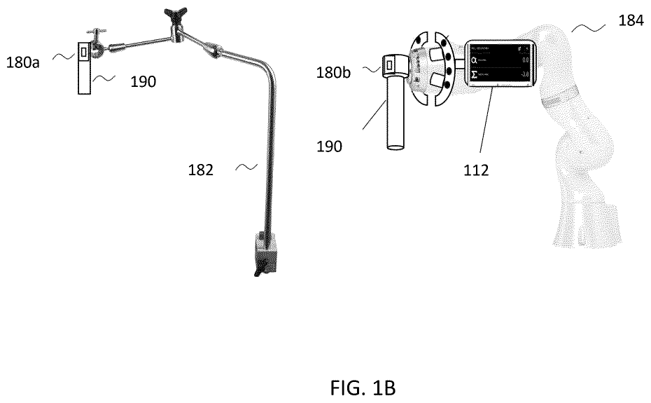

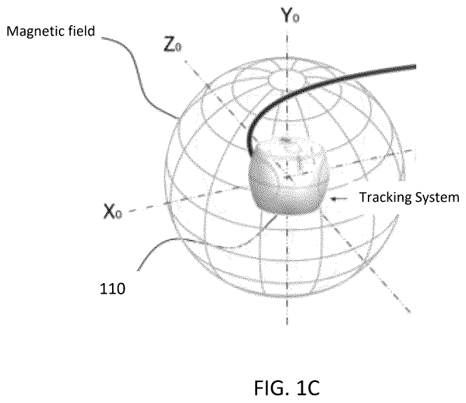

FIG. 1A illustrates a surgical navigation system 100 according to an exemplary embodiment of the subject disclosure. The surgical navigation system 100 can include one or more of a processor 103, joint sensors 102a, 102b, 102c, 102d (collectively 102), markers 104a, 104b, 104c, 104d (collectively 104), surgical instruments 106a, 106b, 106c, 106d, 106e (collectively 106), instrument sensors 108a, 108b, 108c, 108d, 108e (collectively 108), a tracking system 110 (FIG. 1C), a display 112, table arm sensors 180a, 180b (collectively 180), a reference sensor 114, and a network 116. The surgical navigation system 100 uses electromagnetic fields to locate and track joints of a patient and/or medical instruments used during surgery via sensors attached to the patient and/or instrument. In an exemplary embodiment, the surgical navigation system 100 uses electromagnetic fields to locate and track joints of a patient and/or medical instruments in 3D space.

The tracking system 110 emits an electromagnetic field, such as a low intensity, varying electromagnetic field. In an exemplary embodiment, the tracking system 110 can be a magnetic field generator for generating a magnetic field, although in other embodiments the tracking system 110 can include a magnetic field generator 111 for generating a magnetic field as integrated to tracking system 110 body or a separate unit. The magnetic field generator can be e.g., AC-driven, DC-driven, or passive, and defines a measurement volume within which the motion sensors will be tracked.

The surgical navigation system 100 can include one or more joint sensors 102. The joint sensor 102 can provide (e.g., transmit) a signal indicating the position, orientation, and movement of the joint sensor 102 with the magnetic field generated by the tracking system 110. In an exemplary embodiment, when the joint sensor 102 enters the electromagnetic field, a small current can be induced, which can be relayed to the processor 103. In such an embodiment, the processor 103 receives the current provided via the joint sensor 102 and converts the current to a digital signal. The joint sensor 102 can provide the signal to the processor 103 or to the tracking system 110 via a wired transmission or wirelessly. In examples in which the joint sensor 102 provides the signal to the tracker system 110, the tracking system 110 can provide the signal to the processor 103. The digitized signal can be used to calculate the position, orientation, and movement of the joint sensor 102. The position, orientation, and movement of the joint sensor 102 can be correlated to the position, orientation, and movement of the joint of the patient, such as the vertebra of the patient. Alternatively, the tracking system 110 can track the joint sensors within the electromagnetic field by measuring the field's characteristics at different locations. In such embodiments, the tracking system 110 includes both a generator and a tracker integrated body. The position, orientation, and movement data is sent to the display 112, which can display the position, orientation, and movement of the joint sensor 102. The joint sensors are applicable to any joint of the patient, such as a vertebrae, a knee joint, a hip joint, an elbow joint, an ankle joint, a shoulder joint, or a wrist joint of the patient.

The joint sensor 102 can be a 5-degree-of-freedom (5-DOF) sensor or a 6-degree-of-freedom (6-DOF) sensor. The joint sensor 102 are configured to monitor changes in the position, orientation, and movement of each joint sensor 102 (and each respective vertebra 220) in real-time. The joint sensor 102 can transmit the position, orientation, and movement data to the processor 103, tracking system 110, and display 112. In an embodiment, the joint sensor 102 includes a non-ferrous metal and/or include a coil. In an exemplary embodiment, all metal of the joint sensor 102 is a non-ferrous metal.

In an embodiment in which the joint sensor 102 is a 5-DOF sensor, the joint sensor 102 can encompass three rotational degrees of freedom (Roll, Pitch, Yaw) and two translational degrees of freedom (translation along the X and Y axes). Such joint sensor 102 can provide data on roll, pitch, and yaw angles, as well as measuring and transmitting information on the parallel movement distance of the axis.

In an embodiment in which the joint sensor 102 is a six-degree-of-freedom (6-DOF) sensor, the joint sensor 102 can include three rotational degrees of freedom (roll, pitch, and yaw) and three translational degrees of freedom (translation along the X, Y, and Z axes). Such joint sensor 102 provides data on roll, pitch, and yaw angles, along with measuring and transmitting information on the parallel movement distance of the axis. Utilizing a 6-DOF sensor enables a more precise measurement of the position and orientation of the joint sensor 102, marker 104, and corresponding joint (e.g., vertebra 220).

The surgical navigation system 100 can include a marker 104 for attaching the joint sensor 102 to a joint of a patient. In an exemplary embodiment the joint sensor 102 is configured to attach to the marker 104, and the marker 104 is configured to attach to a portion of a patient, such as a joint of a patient. In embodiments the marker 104 can be a pin and the like. One or more markers 104 can attach a plurality of joint sensors 102 to a plurality of vertebrae respectively. For example, the marker 104 can attach at least three joint sensors 102 to at least three respective adjacent vertebra.

The surgical navigation system 100 can include an instrument sensor 108. The instrument sensor 108 can be integrated with an instrument 106 or coupled (e.g., detachably coupled) to the instrument 106. Instruments can include retractors (e.g., hand-held retractors, spinal retractors, tubular retractors), rongeurs (e.g., Kerrison rongeurs, spinal IVD rongeurs, micro-surgical. Rongeurs), bone cutters, drills, spinal punches, rod benders, suction tubes, scalpels and scissors, micro-dissection instruments, and the like. The instrument sensor 108 can provide a signal indicating the position, orientation, and movement of the instrument sensor 108, similar to how the joint sensor 102 provides a signal described herein. The position, orientation, and movement of the instrument sensor 108 can be correlated to the position, orientation, and movement of the instrument 106. In an exemplary embodiment, the instrument sensor 108 can provide the signal to the processor 103, the tracking system 110, and the display 112. The signal may be provided to the processor 103, to the tracking system 110, and/or to the display 112 via a wired transmission or wirelessly.

The instrument sensor 108 can be a 5-degree-of-freedom (5-DOF) sensor or a 6-degree-of-freedom (6-DOF) sensor. The instrument sensor 108 can be configured to monitor changes in the position, orientation, and movement of each instrument sensor 108 (and the respective surgical instrument 106) in real-time. The instrument sensor 108 can transmit the position, orientation, and movement data to the processor 103, the tracking system 110, and the display 112. In an embodiment, the instrument sensor 108 includes a non-ferrous metal. In an exemplary embodiment, all metal of the instrument sensor 108 is a non-ferrous metal.

In an embodiment in which the instrument sensor 108 is a 5-DOF sensor, the instrument sensor 108 will encompass three rotational degrees of freedom (Roll, Pitch, Yaw) and two translational degrees of freedom (translation along the X and Y axes). Such instrument sensor 108 can provide data on roll, pitch, and yaw angles, as well as measuring and transmitting information on the parallel movement distance of the axis. In an embodiment in which the instrument sensor 108 is a six-degree-of-freedom (6-DOF) sensor, the instrument sensor 108 can include three rotational degrees of freedom (roll, pitch, and yaw) and three translational degrees of freedom (translation along the X, Y, and Z axes). Such instrument sensor 108 provides data on roll, pitch, and yaw angles, along with measuring and transmitting information on the parallel movement distance of the axis. Utilizing a 6-DOF sensor enables a more precise measurement of the position and orientation of the instrument sensor 108 and surgical instrument 108.

The surgical navigation system 100 can include a processor 103. The processor 103 can be used to receive and/or process image data of the patient, such as patient-specific image data of the patient. The image data can be data representing 2D or 3D images of the patient, a surgical instrument, and the like. In an exemplary embodiment, the image data can be C-arm image data, which allows surgeons to visualize bone structures, joints, and surgical devices during surgery, although in embodiments the image data can be any image data typically used in surgery or for surgical navigation systems.

The processor 103 can operatively communicate with the rest of the surgical navigation system 100, such as the tracking system 110 and sensors. In an exemplary embodiment, the processor 103 can be used to receive and/or process image data relating to a joint of the patient. The processor 103 can be used to receive and/or process image data and position data relating to the surgical instrument, to receive and/or process image data and position data relating to sensors (e.g., surgical sensor and/or instrument sensor), and the like. The image data of the patient, instrument, and sensors can be 2D image data and/or 3D image data.

The processor 103 can be used to receive data from the joint sensor 102, such as position, orientation, and/or movement of the joint sensor 102 and/or the instrument sensor 108. The processor 103 can update the patient image data using the position, orientation, and/or movement data of the joint sensor 102 and/or the instrument sensor 108. In an embodiment, the processor 103 can update the patient image data by overlaying the position, orientation, and/or movement data of the joint sensor 102 and/or the instrument sensor 108 upon the patient image data.

The processor 103 can determine the size and shape of the joint of the patient using the patient-specific image data. When the image data is 2D, the processor 103 can determine the length and width of the joint via the image data. When the image data is 3D, the processor 103 can determine the length, width, and height of the joint via the patient-specific image data. The processor 103 can determine the shape, contour, holes, additions, indentations, and the like of the joint of the patient via the patient-specific image data.

The processor 103 can be one or more processors, microprocessors, computer processing units (CPUs), graphics processing units (GPUs), neural processing units, physics processing units, digital signal processors, image signal processors, synergistic processing elements, field-programmable gate arrays (FPGAs), sound chips, multi-core processors, and the like. The processor 103 can be operatively in communication with a memory having stored thereon or received therein computer instructions executable by the processor 103 to carry out the functions as described herein.

The surgical navigation system 100 can include a display 112. The display 112 can be used to display text, models, virtual surgical procedures, surgical plans, implants, graphics, images, and the like. In an embodiment, the display 112 can include an LCD display screen, an LED display screen, a projected, holographic, or augmented reality display (such as a heads-up display device or a head-mounted device), and so on. The display 112 can be used to receive, process, and display image data of the patient, such as patient-specific image data (e.g., 2D image data and/or 3D image data) of the patient. The display 112 can be used to receive, process, output and/or display the data provided by the processor, joint sensor 102 and the instrument sensor 108. For example, the display 112 can be used to receive, process, output and/or display data from the joint sensor 102, such as position, orientation, and/or movement of the joint sensor 102 and/or the instrument sensor 108.

As described herein, the processor 103 can update the patient image data (e.g., joint of the patient) by overlaying the position, orientation, and/or movement data of the joint sensor 102 and/or the instrument sensor 108 upon the patient image data. The display 112 can display the position, orientation, and/or movement data of the joint sensor 102 and/or the instrument sensor 108 upon the image data.

The display 112 can display the position, orientation, and/or movement data of the joint of the patient surgical instrument 106, joint sensor 102, and/or the instrument sensor 108. In an embodiment, the display 112 can display the position, orientation, and/or movement data of the joint of the patient, surgical instrument 106, joint sensor 102, and/or the instrument sensor 108 to scale based on the image data as well as other information, such as the model numbers of the instrument 106, instrument sensors 108, and/or joint sensors 102.

The surgical navigation system 100 can include one or more table arm sensors 180, which are depicted in FIG. 1B. A table arm sensor 180a can be attached to a manual articulating table arm, such as the manual articulating table arm 182 with trajectory guide 190. A table arm sensor 180b can be attached to an automatic or semi-automatic articulating arm, such as semi-automatic articulating arm 184 with trajectory guide 190. The table arm sensor 180 can provide a signal indicating the position, orientation, and movement of the table arm sensor 180, similar to how the joint sensor 102 provides a signal described herein. The position, orientation, and movement of the table arm sensor 180 can be correlated to the position, orientation, and movement of the respective table arm 182, 184. In an exemplary embodiment, the table arm sensor 180 can provide the signal to the processor 103, the tracking system 110, and the display 112. The signal may be provided to the processor 103, to the tracking system 110, and/or to the display 112 via a wired transmission or wirelessly. Display 112 can be used to display information relating to table arm sensor 180 similar to how display 112 displays information relating to other sensors, as described herein.

The surgical navigation system 100 can include a reference sensor, such as reference sensor 114. The reference sensor 114 can be detectable within the magnetic field and can be used to register a frame of reference for the joint sensor 102 and the instrument sensor 108. For example, the reference sensor 114 is configured to establish a fixed location and/or frame of reference against from which the surgical sensor 102 and/or instrument sensor 108 are defined.

The surgical navigation system 100 includes a network 116. The network 116 can facilitate communication from one of the devices in the surgical navigation system 100 to one or more other devices within the surgical navigation system 100. For example, the network 116 can facilitate communication between the joint sensors 102, tracking system 110, processor 103, and display 112. The network 116 can facilitate communication from one or more devices to one or more other devices via a direct link or an indirect link. A direct link can include a link between two devices where information is communicated from one device to the other without passing through an intermediary. For example, the direct link can include a Bluetooth™ connection, a Zigbee™ connection, a WIFI Direct™ connection, a near-field communications (NFC) connection, an infrared connection, a wired universal serial bus (USB) connection, an ethernet cable connection, a fiber-optic connection, a firewire connection, a microwire connection, and so forth. In another example, the direct link can include a cable on a bus network.

An indirect link can include a link between two or more devices where data can pass through an intermediary, such as a router, before being received by an intended recipient of the data. For example, the indirect link can include a wireless fidelity (WIFI) connection where data is passed through a WIFI router, a cellular network connection where data is passed through a cellular network router, a wired network connection where devices are interconnected through hubs and/or routers, and so forth. The cellular network connection can be implemented according to one or more cellular network standards, including the global system for mobile communications (GSM) standard, a code division multiple access (CDMA) standard such as the universal mobile telecommunications standard, an orthogonal frequency division multiple access (OFDMA) standard such as the long-term evolution (LTE) standard, and so forth.

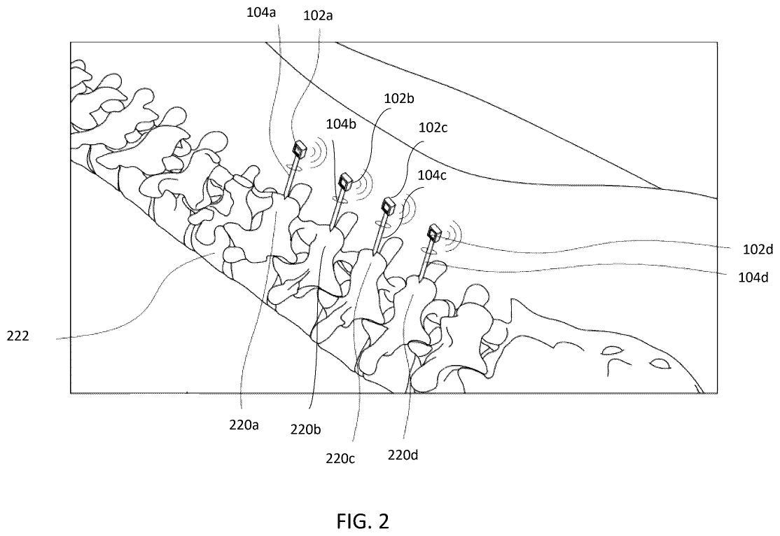

FIG. 2 illustrates a plurality of joint sensors 102 and a corresponding plurality of markers 104 attached to a vertebrae of a patient. Each of the plurality of sensors 102 are attached to a joint of a patient, such as a vertebra 220a, 220b, 220c, 220d (collectively 220) of a patient's vertebrae 222 via respective markers 104. Each of the joint sensors 102 are configured to send a signal indicating the position, orientation, and/or movement of each respective joint sensor 102 relative to each other and the vertebrae. The processor 103 is configured to receive image data of the joint of the patient and overlay the position, orientation, and/or movement of the joint of the patient based on the signal of each of the joint sensors 102 either directly or from the sensor tracking unit 110. By receiving signals from multiple joint sensors 102, the processor 103 can provide a 3D image of all joints in which a joint sensor 102 is attached.

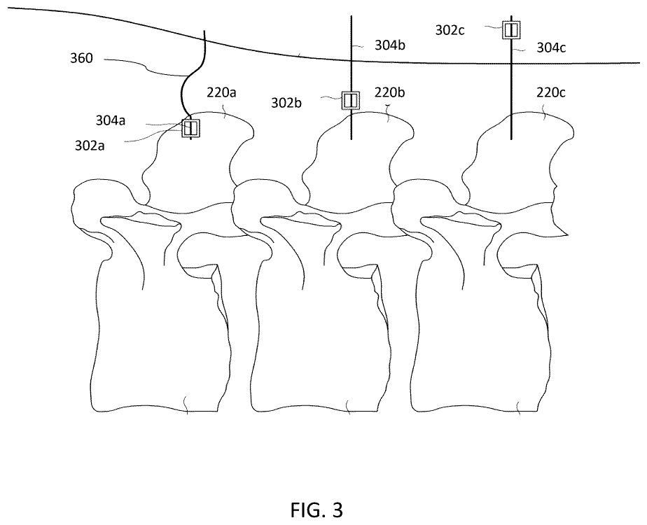

FIG. 3 shows an alternative configuration in which the joint sensors 302a, 302b, 302c (collectively 302) are attached to the vertebra 220 of a patient via a marker (e.g., pin). As shown in FIG. 3, the joint sensor 302a is attached to the vertebra 220a via a pin 304a. A fiber member 360 can be exposed externally from the skin of the patient. For example, once the joint sensor 302a is inserted into an incision of a patient's skin, the fiber member 360 can protrude outward. Such exposure of the fiber member 360 allows a surgeon to determine the position of the joint sensor 302a. In embodiments the fiber member 360 can take the form of a lengthy strand, such as a thread or string.

In embodiments, a joint sensor 302 can be attached to a vertebra of a patient, such as the pin 304b attaching the joint sensor 302b to the vertebra 220b, and the pin 304c attaching joint sensor 302c to vertebra 220c. The joint sensor 302 may be placed about different areas of a marker (e.g., pin), such as the joint sensor 302b being placed on the pin 304b proximate to the vertebra 220b (e.g., below the skin of the patient) and the joint sensor 302c being placed on the pin 304c distal to the vertebra 220c (e.g., above the skin of the patient).

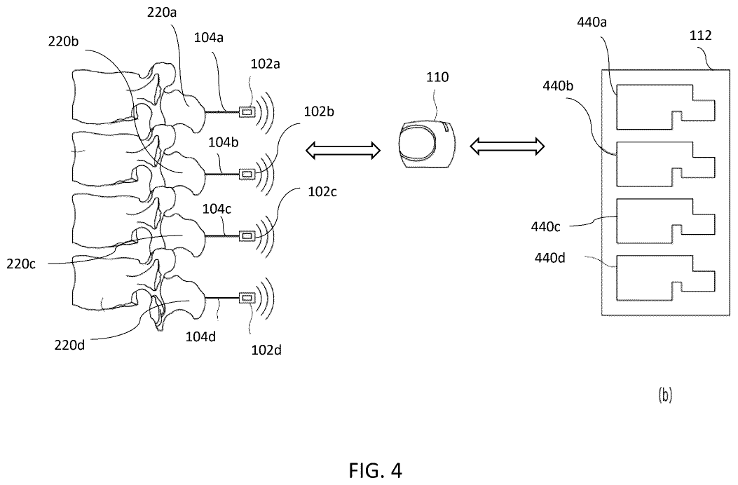

FIG. 4 shows an embodiment of joint sensors 102 attached to the vertebra 220a, 220b, 220c, 220d of a patient via markers 104. Each of the joint sensors 102 induce a signal that is transmitted to the electromagnetic field generator/tracking system 110. As described herein, the signal can be a current that is digitized and thereafter converted to position, orientation, and movement information of each of the joint sensors 102 within the electromagnetic field and relative to the patient's joint. In an embodiment the tracking system 110 can digitize and convert the signal to position, orientation, and movement information of each of the joint sensors 102. In other embodiments the processor 103 can digitize and convert the signal to position, orientation, and movement information of each of the joint sensors 102. In embodiments in which the tracking system 110 digitizes and converts the signal to position, orientation, and movement information of each of the joint sensors 102, the tracking system 110 can send the position, orientation, and movement information to the processor 103. The tracking system 110 and/or the processor 103 can transmit the position, orientation, and movement information to the display 112. The position, orientation, and movement information can be transmitted via network 116.

As shown in FIG. 4, the display 112 shows joint sensor representations 440a, 440b, 440c, 440d (collectively 440) providing a digital representation of the joint sensors 402 in a first position. The joint sensor representations 440 are shown on the display 112 having the same positions and orientations of the corresponding joint sensors 402. As described herein, the shape, size, contour, and the like of the joint representation 440 is based on the vertebra shown on the image data received by the processor 103. For example, the joint representation 440a is shown on the display 112 having the same position and orientation as the corresponding joint sensor 102a (as well as the marker 104a and the vertebra 220a), the joint representation 440b is shown on the display 112 having the same position and orientation as the corresponding joint sensor 102b (as well as the marker 104b and the vertebra 220b), the joint representation 440c is shown on the display 112 having the same position and orientation as the corresponding joint sensor 102c (as well as the marker 104c and the vertebra 220c), and the joint representation 440d is shown on the display 112 having the same position and orientation as the corresponding joint sensor 102d (as well as the marker 104d and the vertebra 220d).

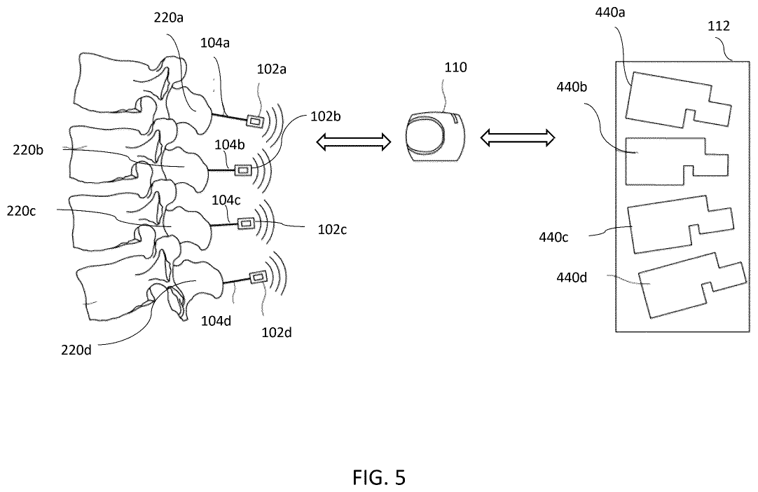

FIG. 5 shows the joint sensors 102 attached to the vertebra 220a, 220b, 220c, 220d of a patient via the markers 104 in a second position that differs from the first position. A comparison of FIG. 4 to FIG. 5 illustrates the tracking of the sensors and joints as the joints are moved between first and second positions.

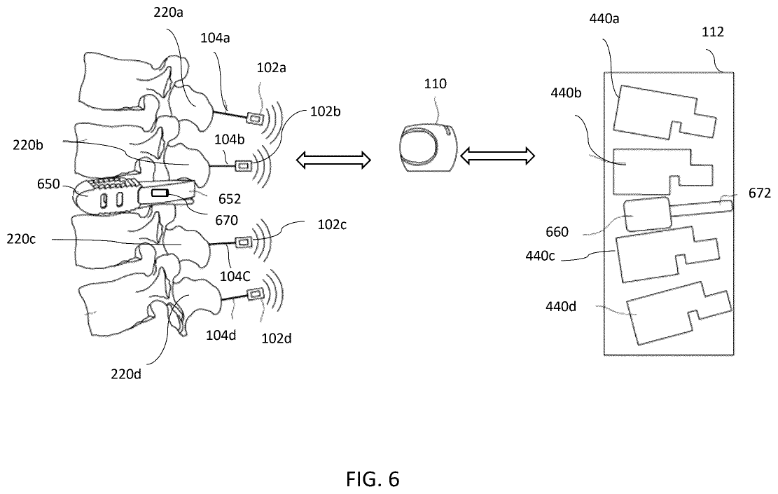

FIG. 6 shows an exemplary embodiment in which an instrument sensor 670 is attached to or integrated with a surgical instrument 652. The surgical instrument 652 can be used to insert an implant (e.g., cage) within a patient, such as within joints of a patient. As shown in FIG. 6, an instrument sensor 670 can be attached to or integrated with the surgical instrument 652. The display 112 shows an implant representation 660 of the implant 650 and an instrument representation 672 of the instrument 652. The implant representation 660 is shown on the display 112 having the same position and orientation as the corresponding implant 650 and the instrument representation 672 is shown on the display 112 having the same position and orientation as the corresponding instrument 652. As described herein, the shape, size, contour, and the like of the instrument representation 672 is based on the surgical instrument 652 and the shape, size, contour, and the like of the implant representation 660 is based on the implant 650. The shape, size, contour, and the like of the instrument representation 672 and the shape, size, contour, and the like of the implant representation 660 is based on image data received by the processor 103.

As shown on FIG. 6, the instrument sensor 670, the instrument 652, and the implant 650 can be used with the joint sensors 102 attached to the vertebra 220a, 220b, 220c, 220d of a patient via the markers 104, as described herein. Each of the instrument sensor 652 and the joint sensors 102 can induce a signal that is sent to the tracking system 110. As described herein, the signal can be a current that is digitized and thereafter converted to position, orientation, and movement information of each of the instrument sensor 652 and the joint sensors 102.

The tracking system 110 and/or the processor 103 can transmit the position, orientation, and/or movement information to the display 112. As shown in FIG. 6, the display 112 shows instrument representation 672 and the implant representation 660 providing a digital representation of the respective instrument 652 and/or surgical implant 650. The instrument representation 672 is shown on the display 112 having the same position and orientation of the corresponding instrument sensor 670 and/or surgical implant 650. The shape, size, contour, and the like of the surgical implant 650 is based the image data received by the processor 103. For example, the instrument representation 660 is shown on the display 112 having the same position and orientation as corresponding instrument sensor 652 and/or surgical implant 650.

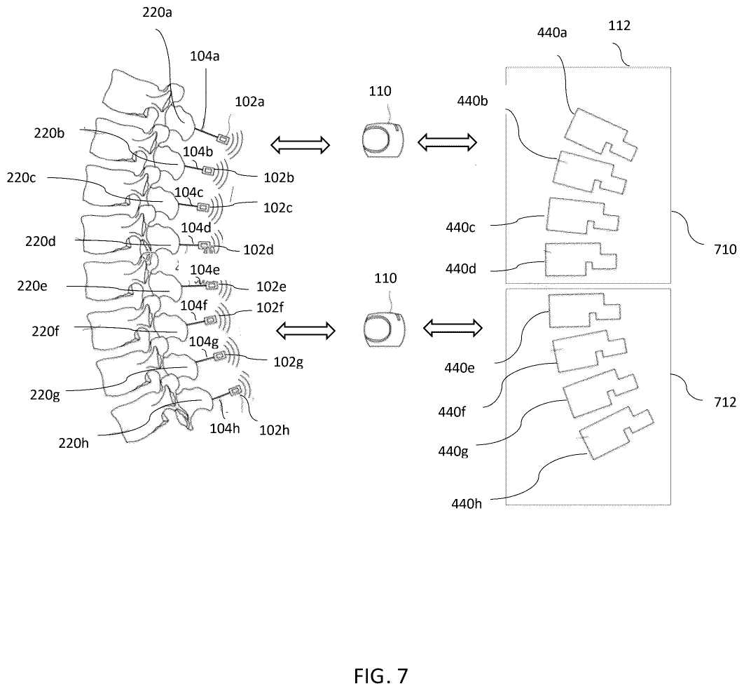

FIG. 7 shows an embodiment in which more than one generator/tracking system 110, such as tracking systems 110a and 110b, are used to generate and/or emit an electromagnetic field. Devices, components, and joints that are similarly shown in FIGS. 4-6 can be referred via similar reference numbers in FIG. 7. As shown in FIG. 7, more than one tracking system 110 can be used to increase the zone of the magnetic field. By increasing the zone of the magnetic field, a larger anatomy of a patient can be captured, additional segments of a patient can be captured, and anatomies of more than one patient can be captured. For example, as shown in FIG. 7, the tracking system 110a is able to generate and/or emit an electromagnetic field that can support joint sensors 102a, 102b, 102c, and 102d that are found within a first zone 710 or segment, and the tracking system 110b is able to generate and/or emit an electromagnetic field that can support joint sensors 102e, 102f, 102g, and 102h that are found within a second zone 712 or segment.

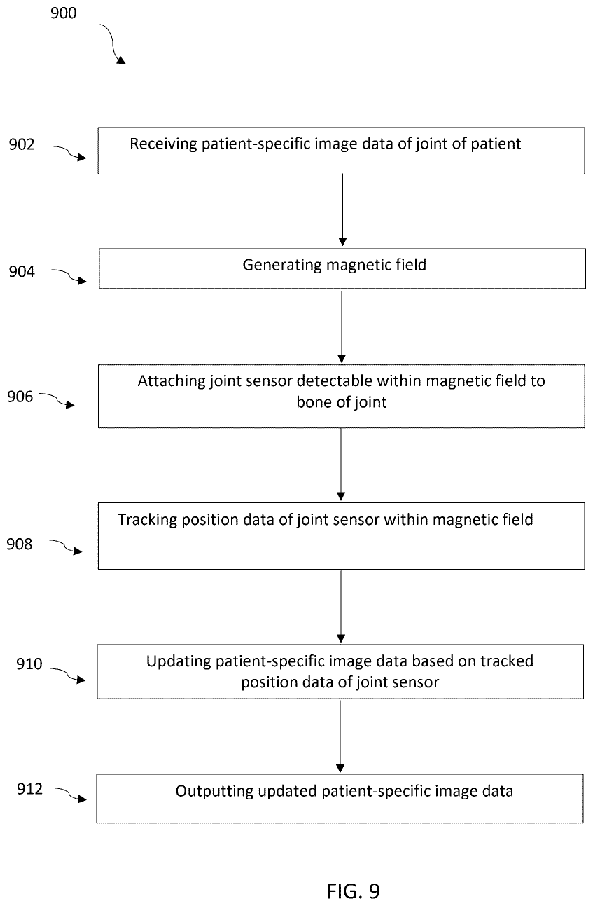

FIG. 9 shows an exemplary embodiment of a surgical navigation method 900 in accordance an exemplary embodiment of the subject disclosure. At Step 902, the patient-specific image data of a joint of a patient is received by a surgical navigation system. The patient-specific image data can be 2D image data, 3D image data, and the like. The patient-specific image data can be produced prior to surgery (e.g., via a C-arm imaging device during surgery preparations) or can be produced during surgery, i.e., the patient-specific image data can be pre-operative image data or intra-operative image data.

At Step 904, a magnetic field is generated. The magnetic field can be generated via a tracking system of the surgical navigation system. In an example, the electromagnetic field can be a low intensity, varying electromagnetic field.

At Step 906, a joint sensor (e.g., joint sensor) detectable within magnetic field is attached to a bone of a joint e.g., a first bone of a joint. At Step 908, position data of the joint sensor can be tracked within the magnetic field. For example, the joint sensor can provide (e.g., transmit) a signal indicating the position, orientation, and movement of the joint sensor. In an example, when the joint sensor enters the electromagnetic field, a small current can be induced, which can be relayed to a processor. The processor can receive the current provided via the joint sensor and convert the current to a digital signal. The joint sensor can provide the signal to the processor via a wired transmission or wirelessly. The digitized signal can be used to calculate the position, orientation, and movement of the joint sensor. The position, orientation, and movement of the joint sensor can be correlated to the position, orientation, and movement of the joint of the patient, such as the vertebra of the patient.

At Step 910, the patient-specific image data can be updated based on the tracked position data of the joint sensor. For example, the processor can update the image data of the patient using the position, orientation, and/or movement data of the joint sensor and/or the instrument sensor. In an embodiment, the processor can update the image data by overlaying the position, orientation, and/or movement data of the joint sensor and/or the instrument sensor upon the image data. At Step 912, the updated patient-specific image data can be output, for example, via a display.

In an embodiment, the surgical navigation system as described herein can be use sensors (e.g., electromagnetic sensors) to view the actual joint (e.g., vertebra) of a patient prior to surgery or during surgery. The surgical navigation system can use the sensors to view the actual surgical instrument or implant to be used within a body of a patient prior to surgery or during surgery. As described herein, the sensors allow the surgeon to view the position, orientation, and/or movement of the joint of a patient, surgical instrument, and implant in real time. As a result, the surgeon can view more accurate representations of the joint, instrument, and implant during surgery, and therefore more confidence in performing such surgeries.

The surgical navigation system 100 can provide one or more of the following advantages over conventional optical sensor-based surgical navigation systems. Unlike the surgical navigation system 100, surgical planning using conventional methods is constrained by reliance on pre-operative scans. The pre-operative scans can present a mismatch between the real-time anatomy of the patient and the pre-operative images shown on a display. In addition, conventional surgical navigation systems based on optical sensors cannot effectively assist with the placement of devices (e.g., pedicle screws) within a joint of the patient because the tracking field of vision gets cluttered using optical sensors. Further, unlike conventional surgical navigation systems using optical sensors, the surgical navigation system 100 can operate without requiring a direct line of sight, as the known natural magnetic field source is unaffected by Electromagnetic Interference (EMI).

Moreover, instruments used in conventional optical sensor-based surgical navigation systems can be heavy and large, which can limit maneuverability and cause the instruments to become difficult to handle and prone to interference with other surgical tools or unintended movements of the surgeon. Further, conventional optical sensor-based surgical navigation systems can require spatial real-estate, such as at least four array spheres being held far apart from each other in a bracket type of frame. The large marker frame brackets can lead to collisions between the hands of the surgeon and the markers attached to both the surgical instrument and the joint (e.g., spine) of the patient. Further still, slight movements of markers during surgery using conventional optical sensor-based surgical navigation systems can necessitate re-registering the pre-operative vertebral image and the sensor position, which can disrupt the surgery.

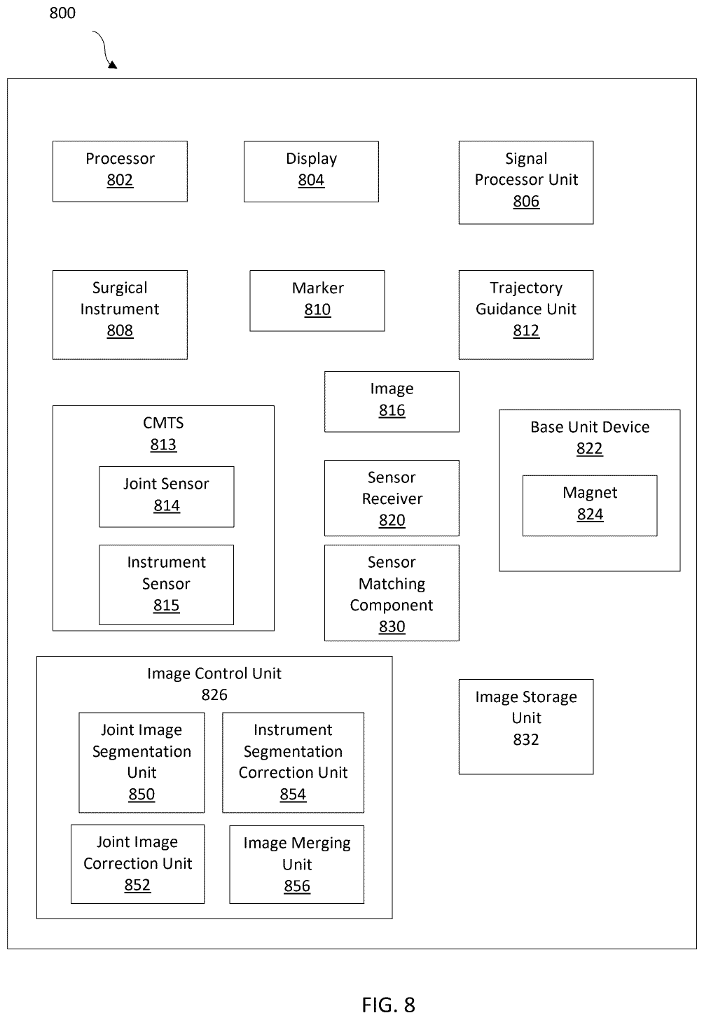

In accordance with another exemplary embodiment, the subject disclosure provides a surgical navigation system 800, as shown in FIG. 8. The surgical navigation system 800 overcomes limitations of conventional navigation systems by employing a cost-effective and real time position tracking sensor system. Surgical navigation system 800 can include the use of a processor 802, a display 804, a signal generation and/or processing base unit 806, surgical instruments 808, anatomy markers 810, trajectory guidance units(s) 812 having attachable compact motion tracking sensors (CMTS 813), or being integrated with CMTS 813. The CMTS 813 can include one or more joint sensors 814 or instrument sensors 815.

The CMTS 813 being compact can minimize the risk of a hand of a surgeon hitting the CMTS 813 during surgery and can minimize the amount of occupied surgical-site access space by tracking elements such as the described optical array sensors. The CMTS 813 being compact can provide more rigid anchoring to more than one anatomical segment with multiple markers, where one marker can be at each anatomical segment. The CMTS 813 can reduce or eliminate inaccuracies of conventional systems and allow the surgical navigation system 800 to accurately reflect the patient's near-real-time individual anatomical segment position, orientation, and movement changes that have occurred during a surgical procedure.

The surgical navigation system 800 can address the limitations of conventional surgical navigation systems by: (1) eliminating a dependency on the line-of-sight requirements associated with optical reflective markers due to the use of non-optical sensor technology; (2) monitoring multiple adjacent anatomical segments due to deployment of multiple CMTS 813; (3) tracking real-time motion and orientation changes in each anatomical segment and instrument; and (4) providing flexibility to attach motion sensors and/or trackers in a wider range of locations such as to the proximal, middle, or distal part of the markers, surgical tool, and/or trajectory guidance units due to its compact size. As a result, the surgical navigation system 800 can accurately measure the actual movement of the joint (e.g., vertebrae) of a patient during surgery.

The surgical navigation system 800 can provide an improvement to joint surgical navigation via use of a non-optical sensor (e.g., CTMS) vs. bulky optical sensor-based systems used in conventional systems. The surgical navigation system 800 can motion and spatial track orientation based on energy field generator/detector/data acquisition-transferring base unit(s); anatomy markers 810 with joint sensors 814; surgical instruments 808 with instrument sensors 815; and/or trajectory guidance unit(s) 812 with CTMS. The position and orientation information can be used to create numerous applications and/or methods, as described herein.

The method of surgical navigation can include one or more of storing 2D or 3D images 816 of the patient's joint; combining the images 816 with one or more markers 810 for tracking the real-time position and orientation of an individual joint (e.g., vertebrae), superimposing the position and orientation of the surgery tool model(s) over the patient's images 816, and/or projecting the potential, current, or desired trajectory of the surgical instrument 808.

An anatomy marker 810 can be equipped with the joint sensor 814 that affixes to the joint (e.g., spine vertebrae) of a patient and continuously measures and transmitting the position and orientation of the marker 810. The anatomy marker 810 can be one or more devices such as a pin, a tag, a removable appendage of an implant, an implant interfacing compact instrument, a clamp forceps, etc. The surgical instrument 808 can include such devices as an awl, drill, tap, probe, screw inserter, etc., and can monitor the real-time position and orientation of the surgical instrument 808 via the instrument sensor 815. Trajectory guidance unit(s) 812 with a CTMS can provide a desired trajectory and/or stop guidance. In an embodiment, the trajectory guidance unit(s) 812 can be a surgical table's articulating arms (manual or electric) with trajectory guiding elements.

A processor 802 can archive 2D or 3D images 816 of a spine captured via a C-arm imaging device 818 during surgery preparations, alongside images 816 of various surgery instruments 808. The CTMS can be attached to or integrated about an anatomical marker 810 (such as pin or tag) and/or a surgical instrument 808 and can include a 5-degree-of-freedom (5-DOF) or a 6-degree-of-freedom (6-DOF) sensor element. Real-time changes in vertebrae orientation and position can be determined and such information can be transmitted to the sensor receiver 820.

Various motion sensor technologies exists in the market including optical, electromagnetic, mechanical, and inertial techniques. In an embodiment, the surgical navigation system 800 can use a base unit device 822 that utilizes a permanent magnet 824. The magnet 824 can be used to generate, receive, and process magnetic field information equipped with a built-in technique. The base unit device 822 can work in conjunction with a motion-detecting sensor/tracker unit(s), such as the CMTS 813. A technique can compare the distorted magnetic field data with the natural magnetic field emitted by the permanent magnet 824. The surgical navigation system 800 can operate without requiring a direct line of sight, as the known natural magnetic field source is unaffected by Electromagnetic Interference (EMI).

An image control unit 826 can subdivide the stored joint (e.g., spine) image data into segments and assign to each segment an identifier (e.g., a numerical identifier). The image control unit 826 can employ correction units to refine the imagery based on data from corresponding motion sensors as well as adjusting surgical tool images. An image merging unit 856 can combine the corrected images to create real-time augmented reality displays. The image control unit 826 can incorporate a sensor matching component 830. The sensor matching component 830 can align each CMTS 813 with a corresponding image 816 of a segment. The alignment can facilitate precise correction of the imagery for each spinal segment by utilizing data from the segment-matched sensors.

A surgical navigation can be performed via one or more of the following steps: A 2D image of the patient's anatomy (such as the spine) can be obtained (e.g., obtained using a C-arm imaging device in the operating room). In an embodiment, a 3D image can be prepared alongside 2D or 3D images for various surgery instruments. A marker 810 having at least one joint sensor 814 (e.g., 5-DOF or 6-DOF sensor) can be affixed to each anatomical segment, such as each vertebra of the patient. An instrument sensor 815 can be attached to a surgical instrument 808. In an embodiment, the CMTS 813 can emit information via a wireless signal, a sound, haptic feedback, light, and the like, to provide orientation and location information of the CMTS 813. The surgical navigation system 800 can determine if the CMTS 813 are aligned/misaligned with the desired trajectory and/or placement of the CMTS 813. In an embodiment, the CMTS 813 and/or marker 810 can encompass a gravitometer for providing absolute orientation. In another embodiment, the CMTS 813 and/or marker 810 can be used for optically detectable geometry or properties for ease of detection for future compensation techniques.

Real-time tracking can include the joint sensor 814 continuously measuring and transmitting real-time information about the posture and position of each anatomical segment of the patient, such as the vertebrae including a joint sensor 814 and the surgical instrument 808 including an instrument sensor 815.

Image correction and AR generation can be provided. In an embodiment, the image correction and AR generation can include the Sensor Information Receiver 820 processing the transmitted measurement information and using the posture and position data from multiple joint sensors 814 associated with one or more joints to generate 2D or 3D images of the spine. 2D or 3D images of the surgical instrument 808 can be adjusted using information from a corresponding instrument sensors 815, and the visual representation of the 2D or 3D images can be corrected. The corrected and tracked images of each joint (e.g., spinal) segment and the surgical instrument 808 can be merged to create an augmented reality display.

In an embodiment, segmentation of the 2D or 3D image of the joint (e.g., spine) can be obtained with a C-arm device. For example, the 2D or 3D image of the joint can be segmented into separate images for each joint segment, identifiers can be assigned to each segment, and the updated 2D or 3D images can be stored. Each joint segment image can be adjusted using orientation and position information from the corresponding joint sensors 814 and combined with the 2D or 3D image(s) of the instrument sensor 815. Augmented reality can be achieved by integrating the real-time merged images of each spinal segment and the surgical instrument.

Prior to the step of displaying the updated 2D or 3D images, each joint sensor 814 of the joint (e.g., vertebral) segment can be matched and registered with a corresponding vertebral image for each motion segment image. Correcting the vertebral segment image can include correcting the corresponding vertebral segment image using matching information between the vertebral segment image and the joint sensor 814, as well as posture and position information of the matched joint sensor 814.

The surgical navigation system 800 can utilize C-arm imaging to capture images of each individual joint (e.g., vertebra). A joint sensor 814 can be attached to each of the various joints (e.g., vertebra). The sensor configuration provides real-time measurement of the orientation and position of each joint segment, providing a detailed picture of the joint condition of the patient during surgery. By accurately aligning the real-time position and posture of the actual joint (e.g., vertebra or vertebrae) with a corresponding augmented reality (AR) image displayed on the Display 804, the surgical navigation system 800 can provide peace of mind for both patients and medical staff.

Unlike conventional surgical navigation systems, the sensors (e.g., joint sensor 814 and instrument sensor 815) used in the surgical navigation system 800 are compact and cost effective, which eliminates non-affordability and the issue of medical staff accidentally colliding with sensor-equipped instruments during surgery. The surgical navigation system 800 overcomes the limitations of conventional navigation systems, which are restricted to navigating spinal screw insertion in spinal fusion surgeries, for example. As a result, the surgical navigation system 800 provides navigation capabilities for a wider range of spinal surgical procedures.

As described herein, the surgical navigation system 800 can include a joint sensor 814, an image storage unit 832, a pin or anatomical marker 810, a surgical instrument 808, an instrument sensor 815, a signal generator/sensor information receiver/processor 806, and an image control unit 826. When the joint sensor 814 is inserted into the patient's skin and secured to the vertebrae, it may include a fiber member exposed externally.

The image storage unit 832 can archive the 2D and 3D images 816 of the patient's spine and models of spinal surgery instruments. These images 816 can be captured using an image device 818 (e.g., C-arm imaging device) within an operating room, with the patient being suitably positioned for the surgical procedure. Various angles of the spinal surgery area are photographed and processed to generate 2D or 3D images of the surgery site, which can be saved for each type of spinal surgery instrument. Depending on the surgery, the patient may be positioned differently in the operating room, such as prone, sitting, side-lying, or standing.

In embodiments in which images (e.g., augmented reality images) use 2D video, the 2D image of the patient's spine and the 2D image modeling the spinal surgery instrument can be stored in the image storage unit 832. For augmented reality, a 3D image can be utilized. In the case of image implementation, a 3D image of the patient's spine and a 3D image of a spinal surgery instrument can be stored in the image storage unit 832.

The joint sensor 814 can be affixed to a joint (e.g., vertebra) of a patient using a marker 810 (e.g., pin) inserted from within the patient's skin. In an embodiment a fiber member can be exposed externally. Upon the insertion of the joint sensor 814 the joint sensor 814 is secured into the incision of the patient's skin and the fiber member can protrude outward. The exposure can allow for a determination of the position or orientation of the joint sensor 814. The fiber member can take the form of a lengthy strand, such as thread or string.

The joint sensor 814 can be attached to each marker 810 or pin. The joint sensor 814 monitors changes in the posture and position of each vertebra in real-time and transmits the data to the signal generator/Sensor Information Receiver/processor 806. As described herein, the 5-DOF sensor encompasses three rotational degrees of freedom (Roll, Pitch, Yaw) and two translational degrees of freedom (translation along the X and Y axes). The 5-DOF sensor provides data on roll, pitch, and yaw angles, as well as measuring and transmitting information on the parallel movement distance of the axis. The 6-DOF sensor includes three rotational degrees of freedom (roll, pitch, and yaw) and three translational degrees of freedom (translation along the X, Y, and Z axes). The 6-degree-of-freedom (6-DOF) sensor provides data on roll, pitch, and yaw angles, along with measuring and transmitting information on the parallel movement distance of the axis. Utilizing a 6-degree-of-freedom (6-DOF) sensor enables more precise measurement of the posture and position of the marker 810 and the surgical instrument 808. By accurately measuring the posture and position of the marker 810, the posture and position of each vertebra to which the marker is attached can be precisely determined. It is preferable to use a 6-degree-of-freedom (6DOF) sensor for the joint sensor 814 and the instrument sensor 815 for precise movement measurement.

The markers 810 can be fixed to the joint (e.g., vertebra or vertebrae) of a patient, and a joint sensor 814 can be attached to the marker 810 (e.g., upper part of the marker 810) to measure the joint. When moving, the movement of each vertebra can be measured in real time. The instrument sensor 815 can be attached to the surgical instrument 808 (e.g., upper part of the surgical instrument 808). The instrument sensor 815 measures and transmits the position, orientation, and movement of the surgical instrument 808 in real time.

2D or 3D images 816 of various surgical instruments 808 (e.g., spinal surgical instruments) can be created and stored in the image storage unit 832. The 2D or 3D images of various surgical instruments can be displayed on the display 804 using the posture and position information transmitted from the instrument sensor 815. The posture and position of the 2D or 3D image of the surgical instrument 808 can be matched. The signal generator/Sensor Information Receiver/processor 806 can receive measurement information transmitted from the joint sensor 814 and the instrument sensor 815.

The image control unit 826 can utilize the posture and position information from each of the received joint sensors 814 to correct the stored spine images in the image storage unit 832 on a segment-by-segment basis. For example, the posture and position of the spine segment images displayed on the display 804 are aligned with the movement of the patient's spine segments. The image control unit 826 can adjust the stored 2D or 3D image of the surgical instrument 808 in the image storage unit 832 based on the posture and position information provided by the instrument sensor 815 through the sensor information receiver 820. As a result, the posture and position of the 2D or 3D image of the surgical instrument 808 can be displayed on the display 804 and can correspond to the movements of the instrument sensor 815 being used by the medical staff during surgery. The image control unit 826 can merge the corrected 2D or 3D image of the spine for each segment and the 2D or 3D image of the surgical instrument 808 and displays the augmented reality image on the display 804 in real time.

For joint (e.g., spine) surgery, an imaging device 818 (e.g., C-arm imaging device) may be utilized while the patient assumes positions such as prone, sitting, side lying, or standing. This imaging device 818 can capture 2D graphic images of the patient's spine from various angles, which are transformed into a 3D graphic image using software techniques. The resultant 3D or 2D spine image can be stored in the image storage unit 832, along with pre-produced 2D or 3D images corresponding to different types of spinal surgery instruments 808, including prosthetics, spinal screws, and prosthetic insertion devices.

Multiple markers 810 can be affixed to adjacent joints of a patient, with each joint having a one or more markers 810. Attached to each marker 810 is a joint sensor 814, which includes a 5-degree-of-freedom (5-DOF) sensor or a 6-degree-of-freedom (6-DOF) sensor. The sensors can measure real-time changes in the posture and position of each vertebra and transmit the information to the sensor information receiver 820.

The image control unit 826 consists of several components, including a joint image segmentation unit 850, a joint segment correction unit 852, an instrument correction unit 854, an image merging unit 856, and a sensor matching unit 858.

The joint image segmentation unit 850 can divide the 2D or 3D spine image stored in the image storage unit 832 into images for each spinal segment, assigning an identifier to each segment, and storing them accordingly. The joint image segmentation unit 850 can divide the 2D or 3D joint image into segments corresponding to the joint. For example, the joint image segmentation unit 850 can divide the 2D or 3D spine image into segments corresponding to the spine. To segment and process images for each segment, machine learning-based image segmentation techniques can be employed to learn the characteristics of each joint (e.g., spinal) segment. Alternatively, techniques for identifying the joint segment based on its characteristics may be used for segmentation.

The joint segment correction unit 852 can utilize posture and position information from the joint sensor 814, corresponding to each segmented joint image stored by the joint image segmentation unit 850, to rectify any discrepancies. As a result, the posture and position of the image for each spinal segment will accurately reflect the patient's real-time movement.

The instrument correction unit 854 can use posture and position data from the instrument sensor 815, corresponding to the 2D or 3D image of the surgical instrument stored in the image storage unit 832, to rectify the image. In an embodiment, rectifying the image in this way ensures that the posture and position of the 2D or 3D image of the surgery instrument align in real time with the movements of the actual surgical instrument 808 being used by medical staff.

The image merging unit 856 merges the corrected images of each spinal segment from the joint segment correction unit 852 and the corrected image of the surgical instrument from the instrument correction unit 854 in real time, displaying an augmented reality image on the display 804. The position and posture of the image for each spinal segment displayed on the display 804 accurately corresponds to the real-time position and posture of each segment in the patient's spine. Likewise, the position and posture of the image of the surgical instrument 808 displayed on the display 804 match the actual movements of the spinal surgery tool being utilized by medical staff.

The sensor matching unit 858 aligns and registers each joint sensor 814 with the image of each spine segment divided by the joint image segmentation unit 850.

The joint segment correction unit 852 utilizes information from both the sensor matching unit 858 and the joint sensor 814 to match the image of each spine segment, along with posture and position data from the matched joint sensor 814. As a result, the joint segment correction unit 852 corrects the posture and position of images in real time.

The sensor information receiver 820 receives information transmitted by the joint sensor 814 and the instrument sensor 815 and transmits this data to the image control unit 826.

The spine surgical navigation system 800 detects each joint (e.g., vertebra) of the patient using the joint sensor 814 affixed to each joint. When the patient's joint moves, the movement is measured, and the data is transmitted to the sensor information receiver 820, which can transmit the data to the image control unit 826. The image control unit 826 can generate an augmented reality image that responds in real time to the movement of the patient's joint and displays it on the display 804, ensuring alignment between the augmented reality image and the actual movement of the vertebrae.

The surgical navigation system 800 can use the surgical instrument 808 to connect two neighboring intervertebral bodies during intervertebral body fusion surgery, and accurately measure and reflect the movement and rotation of each vertebra in real time in the augmented reality image. As a result, a problem of a mismatch between the augmented reality image and the actual shape of the vertebrae will be alleviated, thereby providing reassurance to both patients and medical staff.

The surgical navigation system 800 can detect each joint (e.g., vertebra) using the joint sensor 814 affixed to a joint. When the patient's joint moves, the movement is measured and the data is transmitted to the signal generator/Sensor Information Receiver/processor 820, which can transmit the data to the image control unit 826. The image control unit 826 can generate an augmented reality image that responds in real-time to the movement of the patient's joint and displays the joint on the display 804, which help ensures alignment between the augmented reality image and the actual movement of the joint.

A surgical navigation method 1000 using the surgical navigation system 800 described herein can include the following steps. At Step 1002, 2D or 3D images of the joint can be taken using a C-arm imaging device and 2D or 3D images of surgical instruments according to the surgical method adopted by the patient in the operating room. At Step 1004, a joint sensor 814 (e.g., a 5-degree-of-freedom (5DOF) sensor or a 6-degree-of-freedom (6DOF) sensor) can be affixed to one or more (e.g., each) joints of the patient.

At Step 1006, the joint sensor 814 measures and transmits the position, orientation, and movement of the joint in real-time, while the instrument sensor 815 attached to the surgical instrument 808 measures and transmits the posture and position of the surgical instrument 808 in real-time. At Step 1008, the sensor information receiver 820 receives the measurement information transmitted by the joint sensor 814 and the measurement information transmitted by the instrument sensor 815.

At Step 1010, the image control unit 826 corrects the 2D or 3D joint image for each joint segment using the posture and position information of multiple joint sensors 814 received from the Sensor Information Receiver 820, and corrects the 2D or 3D surgical tool image using the posture and position information. The image control unit 826 corrects the 2D or 3D spinal image for each joint segment using the posture and position information of multiple joint sensors 814 received from the Sensor Information Receiver 820. The image control unit 826 corrects the 2D or 3D surgical tool image using the posture and position information of the instrument sensor 815. The image control unit 826 merges the corrected 2D or 3D joint image for each joint segment and the corrected 2D or 3D surgical instrument image to display an augmented reality image.

At Step 1012, the image control unit 826 divides the 2D or 3D joint images captured using a C-arm into images for each joint segment, assigns numbers to each segment, and stores the images. At Step 1014, the image control unit 826 uses the posture and position information of the joint sensor 814 corresponding to each segmented joint image and adjust the images for each joint segment. The image control unit 826 utilizes the posture and position information of the instrument sensor 815 corresponding to 2D or 3D images of surgical instruments 808 and adjusts the images of the instruments.

At Step 1016, the adjusted images of each joint segment and the corrected images of the surgical instruments 808 are combined (e.g., combined in real-time) to display augmented reality images. In an embodiment, each joint sensor 814 can be registered and matched to each segmented joint image. The step of adjusting the segmented spinal images can include correcting the corresponding segmented joint images using matching information between the segmented joint images and the joint sensor 814, as well as the posture and position information of the matched joint sensor 814. The surgical navigation system 800 utilizes joint sensors 814 attached to each joint, which provides individual posture and position measurement of each joint segment during surgery. Real-time and accurate alignment of the actual position and posture of the patient's joint can be provided during surgery with the position and posture of the augmented reality (AR) images displayed on the display 804.

It will be appreciated by those skilled in the art that changes could be made to the various aspects described above without departing from the broad inventive concept thereof. It is to be understood, therefore, that the subject application is not limited to the particular exemplary embodiments disclosed, but it is intended to cover modifications within the spirit and scope of the subject application as defined by the appended claims.

I/We claim:

1. A surgical navigation system comprising:
a tracking system having a magnetic field generator for generating a magnetic field;
a joint sensor detectable within the magnetic field;
an instrument sensor detectable within the magnetic field; and
a processor configured to:
receive patient-specific image data of a joint of a patient,
receive position data from the joint sensor,
receive position data from the instrument sensor,
update the patient-specific image data based on one of the received position data from the joint sensor and the instrument sensor, and
output the updated patient-specific image data.

2. The surgical navigation system of claim 1, wherein each of the joint sensor and the instrument motion sensor are configured to emit a signal.

3. The surgical navigation system of claim 2, wherein the processor is further configured to:
receive the signal emitted from at least one of the joint sensor and the instrument sensor; and
convert the received signal emitted from the at least one of the joint sensor and the instrument sensor to position data.

4. The surgical navigation system of claim 1, wherein the joint sensor includes a marker.

5. The surgical navigation system of claim 4, wherein the marker comprises a pin.

6. The surgical navigation system of claim 1, wherein the joint is a vertebrae, a knee joint, a hip joint, an elbow joint, an ankle joint, a shoulder joint, or a wrist joint.

7. The surgical navigation system of claim 1, further comprising a reference sensor detectable within the magnetic field for registering a frame of reference.

8. The surgical navigation system of claim 1, wherein the joint sensor and the instrument sensor comprise a non-ferrous metal.

9. The surgical navigation system of claim 1, wherein the processor is further configured to:
receive image data of a surgical instrument; and
update the patient-specific image data based on the received image data of the surgical instrument.

10. A method for performing surgical navigation comprising:
receiving patient-specific image data of a joint of a patient;
generating a magnetic field;
attaching a joint sensor detectable within the magnetic field to a bone of the joint;
tracking position data of the joint sensor within the magnetic field;
updating the patient-specific image data based on the tracked position data of the joint sensor; and
outputting the updated patient-specific image data.

11. The method of claim 10, wherein the joint is a vertebrae.

12. The method of claim 11, further comprising attaching a plurality of joint sensors to a plurality of vertebra respectively.

13. The method of claim 12, further comprising:
tracking position data of each of the plurality of vertebra; and
updating the patient-specific image data based on the tracked position data of at least two of the plurality of vertebra.

14. The method of claim 11, further comprising attaching at least three joint sensors respectively to at least three adjacent vertebra.

15. The method of claim 11, further comprising attaching a reference sensor to a predetermined vertebra of the patient, and attaching at least three joint sensors respectively to at least three vertebra adjacent the predetermined vertebra.

16. The method of claim 10, further comprising attaching the joint sensor to the joint via a marker.

17. The method of claim 10, further comprising:
receiving image data of a surgical instrument;
receiving position data from an instrument sensor; and
updating the patient-specific image data based on the received image data of the surgical instrument and the received position data from the instrument sensor.
